(19) United States
(12) Patent Application Publication
(10) Pub. No.: US 2010/0104613 A1
(43) Pub. Date: Apr. 29, 2010

(54) ANTIPERSPIRANT OR DEODORANT COMPOSITIONS

(75) Inventors: Catrin Sian Chan, Leeds (GB); Martin Peter Cropper, Bebington (GB); Kevin Ronald Franklin, Bebington (GB); Simon Anthony Johnson, Bebington (GB); Robert McKeown, Bebington (GB)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/605,598

(22) Filed: Oct. 26, 2009

(30) Foreign Application Priority Data
Oct. 27, 2008 (EP) EP08167669

(51) Int. Cl. A61K 8/11; A61K 8/18; A61Q 15/00

(52) U.S. Cl. 424/401; 424/65

(57) ABSTRACT

It is desirable for antiperspirant or deodorant roll-on compositions to be able to deliver fragrance over an extended period of time after application, but previously contemplated encapsulates were based on starch or similar water-soluble or dispersible shell materials, rendering them ineffective in aqueous emulsions.

Capsules of fragrances in cross-linked coacervated gelatin shells satisfying defined particle size, shell thickness and hardness criteria enable roll-on compositions to be incorporated into aqueous antiperspirant or deodorant compositions encapsulated fragrance that can be released after the composition has been topically applied to skin.

The present invention relates to antiperspirant or deodorant compositions and in particular to aqueous emulsions delivered delayed perfume release.

Antiperspirant compositions comprising encapsulated fragrance are known in the art. Most of these compositions comprise moisture-sensitive encapsulates, such as those based on gum arabic or gum acacia, starch or certain modified starches, rather than the water-insoluble, shear-sensitive encapsulates employed in the present invention.

WO2006/056096 (Givaudan SA) discloses shear-sensitive encapsulates, largely focussing on their use in fabric conditioner compositions. Amongst the fabric conditioner examples, there is also disclosed as Example 9 an anhydrous antiperspirant composition, comprising gelatin capsules containing 20% fragrance. This prior art is silent concerning antiperspirant compositions comprising capsules having higher levels of encapsulated fragrance and lower levels of encapsulating shell.

It is an object of the present invention in at least some embodiments to ameliorate or overcome one or more of the problems of incorporating fragrances into antiperspirant or deodorant compositions.

It is a further object of some or other embodiments of the present invention to devise aqueous antiperspirant or deodorant compositions that enable triggered release of fragrance over an extended period after application of the composition onto skin.

According to one aspect of the present invention, there is provided an antiperspirant or deodorant composition in the form of an oil-in-water emulsion comprising:
a continuous aqueous phase in which is dissolved or dispersed an antiperspirant or deodorant active;
a dispersed oil phase;
a nonionic emulsifier or mixture of emulsifiers,
a dispersed particulate shear sensitive encapsulated perfume, and optionally a thickener for the continuous phase
   wherein the perfume encapsulates have a shell of cross-linked gelatin coacervate having a thickness of from 0.25 to 9 μm and providing from 10 to 40% by weight of the capsules, a volume average particle diameter of from 25 to 70 μm, a ratio of shell thickness to the average particle diameter in the range of from 1:5 to 1:120, and a Hysitron hardness in the range of from 1.5 MPa to 50 MPa.

By the employment of the present invention, it is possible to deposit on skin a residual fraction of shear-sensitive encapsulated perfume particles, having a high content of fragrance oil, that can be ruptured by the passage of a garment across the surface of the skin or by the movement of one area of skin relative to another, such as in the underarm, at a time when sweating is or is not occurring or irrespective of whether sweating has occurred. Advantage is accordingly taken of the sensitivity of such a capsule on the skin surface to be ruptured by relative movement of garment or skin to skin. The presence of the optionally thickened or gelled liquid carrier enables the significant fraction of the capsules to be so deposited from conventional contact applicators or from conventional aerosol dispensers. This enables improved masking of malodour and enhanced perception of fragrance over a prolonged period.

The incorporation of encapsulates having a low content of shell material (10 to 40% by weight) enables high perfume loadings to be achieved, but can result in instability. The present inventors have found that by careful selection of the parameters of the encapsulates and careful formulation with one or more non-ionic surfactants, oil-in-water emulsion antiperspirant or deodorant compositions may be attained in which the perfume encapsulates show good storage stability, but are still able to deliver substantial perfume for a prolonged time after application.

According to a second aspect of the present invention, there is provided the use of a composition according to the first aspect that simultaneously a) prevents or reduces localised sweating by topical application of a composition according to the first aspect and b) prolongs perception of perfume or masks body-generated malodour, even when sweating is not occurring or irrespective of whether sweating has occurred.

The term “emulsion” herein simply requires the oil to be dispersed non-homogeneously within a continuous aqueous phase with emulsifier or emulsifiers present at the interface between the oil and the aqueous phase. It includes compositions in which the oil is present as dispersed droplets.

The present invention relates to the incorporation into emulsion antiperspirant or deodorant roll-on compositions of shear sensitive encapsulated perfume capsules, the term capsules herein including microcapsules and encapsulates. Shear sensitive herein contemplates that the capsule is capable of releasing its perfume contents by rubbing of the upper arm forwards or backwards across in contact with the chest wall whilst remaining in contact with it or by the rubbing of clothing worn on the upper arm or chest likewise rubbing across skin in the upper arm or armpit to which the antiperspirant composition has been applied. The shear-sensitive capsules may alternatively be termed “friction-sensitive” or “pressure-sensitive”.

The encapsulating material for the shear-sensitive capsules is water-insoluble, in order to survive in the largely aqueous environment typical of the compositions of the present invention. It is also necessary for the capsules not to be water-sensitive, i.e., not to breakdown or rupture merely because water is present.

The encapsulating material for the shear-sensitive capsules herein is desirably selected from cross-linked gelatin. One encapsulation process suitable for forming shear sensitive capsules is often called complex coacervation, which has been described, for example, in U.S. Pat. No. 6,045,835 and which process description is herein incorporated. In such a process, an aqueous solution of a cationic polymer, commonly gelatin or a closely related cationic polymer, is formed at an elevated temperature that is high enough to dissolve the gelatin, commonly at least 40° and in many instances it is unnecessary to exceed 70° C. A range of 40 to 60° C. is very convenient. The solution is typically dilute, often falling in the range of from 1 to 10% w/w and particularly from 2 to 5% w/w. Either before or after dissolution of the gelatin, an oil-in-water emulsion is formed by the introduction of a perfume oil, optionally together with a diluent oil if desired.

A polyanion or like negatively charged polymer is introduced and the composition diluted until a pH is attained of below the isoelectric point of the system, such as below pH5, and particular from pH3.5 to pH 4.5, whereupon a complex coacervate forms around the dispersed perfume oil droplets. The polyanion commonly comprises gum arabic or a charged carboxymethyl cellulose derivative, such an alkali metal salt, of which sodium is the most commonly mentioned example.

The resultant shell is subsequently cross linked, with a short chain aliphatic di-aldehyde, for example a C₄ to C₆ dialdehyde, including in particular glutaraldehyde. The cross linking step is commonly conducted at a temperature of below ambient such as from 5 to 15° C., and particularly in the region of 10° C. Representative weights and proportions of the reactants and of suitable operating conditions are shown in Examples 1, 2 or 3 of the aforementioned U.S. Pat. No. 6,045,835. The skilled man by suitable selection of the parameters within the general process outlined therein is well capable of producing capsules having a volume average particle size in the range of from 30 to 100 μm, particularly up to 75 μm and especially 40 to 60 μm.

A second encapsulation method that is likewise suitable for forming encapsulated perfumes in which the shell comprises cross-linked coacervated gelatin comprises variations of the above process, as contemplated in WO2006/056096. In such variations, microcapsules comprising a blank hydrogel shell are first formed in a dry state and brought into contact with an aqueous or aqueous/alcoholic mixture of a fragrance compound, commonly diluted with a diluent oil. The fragrance compound is transported through the hydrogel shell by aqueous diffusion and is retained inside. If desired, the resultant fragrance-containing microcapsules can be employed without drying, being a paste or liquid dispersion, or can be dried to a powder, which for practical purposes is anhydrous. Although selection of the ratio of fragrance oil to diluent oil is at the discretion of the producer, and may be varied over a wide range, the ratio is often selected in the range of from 1:2 to 1:1, and particularly 3:4 to 1:1, for fragrance:diluent oils.

The proportion of shell material to core perfume oil is at the discretion of the producer, and is attainable by appropriately varying the proportions of the ingredients in the emulsion. It is essential for the shell material to constitute from 10 to 40% of the capsules, and particularly from 12 to 25% by weight of the capsules. By varying the proportions of shell and core, the physical strength of the shell can be varied (for capsules of the same volume average particle size). Accordingly, capsules having the desired combination of characteristics can be selected.

In some preferred embodiments of the present invention, the fragrance oil constitutes from 70 to 85% by weight of the encapsulates and in such embodiments, the balance is provided by the shell.

In other preferred embodiments, the fragrance oil is present together with an oil diluent, for example providing from 25 to 75% by weight of the oil mixture held within the shell, and especially from 40 to 60% by weight. Desirably in such embodiments, the shell constitutes from 12 to 25% by weight of the encapsulates. In certain of such preferred embodiments, the fragrance constitutes from 35 to 50% by weight of the encapsulates, and is complemented by 35 to 50% by weight of diluent oil. If desired, in yet other embodiments, the composition contains some of the encapsulates that contain diluent oil and others that do not, the weight ratio of the two sets of encapsulates being selected in the range of from 25:1 to 1:25 at the discretion of the producer.

It is preferred for the volume average particle diameter (size) of the capsules to be at least 40 μm and in many desirable embodiments is up to 60 μm in diameter. Herein, unless otherwise indicated, the volume average particle diameter of the encapsulates (D[4,3]) is that obtainable using a Malvern Mastersizer, the encapsulates being dispersed in cyclopentasiloxane (DC245) using a dispersion module mixer speed of 2100 rpm. Calculations are made using the General Purpose model, assuming a spherical particle shape and at Normal calculation sensitivity. The shell thickness can be measured by solidifying a dispersion of the capsules in a translucent oil, cutting a thin slice of the solid mass and using a scanning electron microscope to obtain an image of cut-through individual capsules, thereby revealing the inner and outer outline of its annular shell and hence its thickness.

The shell thickness of the microcapsules tend to increase as the particle size increases. The shell thickness accordingly, often ranges mainly within the thickness range of from 0.25 to 9 μm and for many desirable capsules having shells made from coacervated gelatin, at least 90% by volume of the capsules have shells of up to 2.5 μm thickness. Desirably, at least 95% by volume of the capsules have a shell thickness of at least 0.25 μm. The average shell thickness of microcapsules desirably employed herein is up to 1.5 μm. The same or other suitable gelatin coacervate capsules have an average shell thickness of at least 0.4 μm. For capsules of diameter up to 40 μm, the shell thickness is often below 0.75 μm, such as from 0.25 to <0.75μm whereas for particles of at least 40 μm the shell thickness is often from 0.6 to 2.5 μm.

The fragrance-containing capsules for incorporation in the invention antiperspirant compositions are commonly selected having a ratio of volume average diameter:average shell thickness in the range of from 10:1 to 100:1 and in many desirable such capsules in the range of from 30:1 or 40:1 to 80:1.

By virtue of the particle size and the shell thickness of the capsules, the average % volume of the core containing the fragrance oils and any diluent oil, if present, often falls within the range of from 50 to 90%, and in many embodiments from 70 to 87.5%.

The hardness of the capsules, as measured in a Hysitron Tribo-indenter, is an important characteristic that enables them to be incorporated effectively in the invention formulations, retaining the capability of being sheared by frictional contact between skin and skin or clothing. The hardness is desirably in the range of from 0.5 to 50 MPa and especially from 2.5 or 5 up to 25 MPa, and in many embodiments is up to 10 MPa. In certain preferred embodiments, the hardness is in the range of from 3.5 to 5.5 MPa.

A further parameter of interest in relation to the capsules in the instant invention, and particularly their capability to be sheared by friction in the compositions and process of the instant invention, is their “Apparent Reduced Elastic Modulus (Er). Desirably, Er falls within the range of from 20 to 35 MPa, and in many convenient embodiments, in the range of from 22 to 30 MPa.

Measurements of Hysitron hardness (H) and Apparent Reduced Elastic Modulus (Er) are made in the following manner.

Having appropriately mounted a given capsule, the head of the Tribo-indenter, fitted with a Berkovich tip (a three-sided pyramid) compresses the capsule. The instrument is programmed to perform an indent by compressing the sample with an initial contact force of 75 N, for 10 seconds, followed by a position hold stage for 1 second and a decompression stage for 10 seconds. The instrument achieves a very small load (typically around 15-30 μN). The Hysitron Hardness (MPa) and Apparent Reduced Elastic Modulus (also in MPa) are calculated from the relaxation stage of the force deflection data using the following equations.

$$H = \frac{W}{A}$$

W=Compressive force

A=Contact Area (A≈24.56 h_{c}²)

$$Er = \frac{\sqrt{π}}{2 γ} \frac{S}{\sqrt{A}}$$

S=Contact Stiffness (dW/dhₜ)

hₜ=Total Penetration Depth

γ=1.034

$${h}_{c} = {h}_{t} - κ \frac{W}{S}$$

K=3/4

h_{c}=Contact Depth.

By control of the manufacturing process conditions, the resultant dry capsules having the characteristics specified in the ranges or preferred ranges for particles size and mean diameter described herein can be obtained.

The shear sensitive encapsulates can be employed in the antiperspirant compositions in an amount at the discretion of the manufacturer. Commonly, the amount is at least 0.05%, in many instances at least 0.1% and often at least 0.3% by weight of the composition. Usually, the amount is up to 5%, desirably up to 4% and in many instances is up to 3% by weight of the composition. A convenient range is from 0.5 to 2.5% by weight of the composition.

The perfume oil employable herein in the shear sensitive capsules, and/or other capsules and/or non-encapsulated can be selected as is conventional to attain the desired aesthetic result, and comprises usually a blend of at least 5 components, and often at least 20 components. The components can be synthetic or natural extractions, and, in the case of natural oils or oils produced to mimic natural oils, are often mixtures of individual perfume compounds. The perfume oil can comprise, inter alia, any compound or mixture of any two or more such compounds coded as an odour (2) in the Compilation of Odor and Taste Threshold Values Data edited by F A Fazzalari and published by the American Society for Testing and Materials in 1978.

Often, though not exclusively, the perfume compounds acting as perfume components or ingredients in blends have a ClogP (octanol/water partition coefficient) of at least 0.5 and many a ClogP of at least 1. Many of the perfume components that are employable herein can comprise organic compounds having an odour that is discernible by humans that are selected within the chemical classes of aldehydes, ketones, alcohols, esters, terpenes, nitrites and pyrazines. Mixtures of compounds within classes or from more than one class can be blended together to achieve the desired fragrance effect, employing the skill and expertise of the perfumer. As is well known, within the same class, those compounds having a lower molecular weight, often up to about 200, tend to have a lower boiling point and be classified as “top notes”, whereas those having a higher molecular weight tend to have a higher boiling point and be classified as middle or base notes. The distinction, though, is to some extent an arbitrary simplification, because the fragrance oils form a continuum and their characteristics are not significantly different close to on either side of an arbitrary boundary such as a boiling point of 250° C. or 275° C. Herein, the perfume can comprise any blend of oils boiling at below 250° C. (such as in the range 1 to 99% or 4 to 96%, 10 to 90% or 25 to 60%) with the balance provided by compounds having a boiling point above 250° C. The perfumer recognises that the lower boiling point compounds tend to evaporate more quickly after exposure, whereas higher boiling point compounds tend to evaporate more slowly, so that the desired aesthetic effect can be achieved by selecting the proportions of the faster and slower compounds—the faster providing an instant “hit” whilst the slower providing a longer lasting impact. It will also be recognised that a term such as high impact has also been used to describe low boiling point perfume compounds. The properties of the compound stay the same irrespective of whether they are called high impact or top note ingredients.

A further characteristic of a perfume compound is its odour detection threshold (ODT). Some perfume oils are much more easily detected by the human nose than others, but it is a very subjective measurement and varies considerably depending on the way that testing is performed, the prevailing conditions and the make-up of the panel, e.g. age, gender and ethnicity. As a qualitative means of differentiating between the aesthetic attributes of compounds, and enabling the perfumer to choose ingredients that are detected relatively easily, the ODT represents a useful guide, but quantitatively is more dubious.

Some perfume raw materials have a boiling point of less than, or equal to, 250° C., including some which are generally known to have a low odour detection threshold. Others within said list of perfume raw materials have a boiling point of greater than 250° C. of which some are also generally known to have a low odour detection threshold.

Alternatively or additionally, the fragrance incorporated into the capsules can comprise one or a mixture of perfume essential oils, either mixed with each or and/or with synthetic analogues and/or one or more individual perfume compounds, possibly extracted from blossom, leaves, seeds fruit or other plant material. Oils which are herein contemplated include oils from:

Bergamot, cedar atlas, cedar wood, clove, geranium, guaiacwood, jasmine, lavender, lemongrass, lily of the valley, lime, neroli, musk, orange blossom, patchouli, peach blossom, petotgrain, pimento, rose, rosemary and thyme.

It will be recognised that since naturally derived oils comprise a blend in themselves of many components, and the perfume oil commonly comprises a blend of a plurality of synthetic or natural perfume compounds, the perfume oil itself in the encapsulate does not exhibit a single boiling point, ClogP or ODT, even though each individual compound present therein does.

If desired, the composition can include one or more perfume ingredients that provide an additional function beyond smelling attractively. This additional function can comprise deodorancy. Various essential oils and perfume ingredients, for example those passing a deodorant value test as described in U.S. Pat. No. 4,278,658 provide deodorancy as well as malodour masking.

The instant invention employs an effective concentration of an antiperspirant or deodorant active, which is say a concentration that is sufficient to reduce or control sweating or reduce or eliminate body malodour. In many desirable embodiments, the composition contains at least 1% antiperspirant active, and preferably at least 5% and often is at least 10%. Commonly, the concentration of the antiperspirant active is not higher than 30%, and in many practical embodiments is not higher than 25.5%, %s herein being by weight based on the composition unless otherwise stated. A preferred concentration range for the antiperspirant active is from 10 to 20%.

The antiperspirant active is conveniently an astringent aluminium and/or zirconium salt, including astringent inorganic salts, astringent salts with organic anions and complexes of such salts. Preferred astringent salts include aluminium, zirconium and aluminium/zirconium halides and halohydrate salts, such as especially chlorohydrates. Activated chlorohydrates can be incorporated, if desired.

Aluminium halohydrates are usually defined by the general formula Al₂(OH)ₓQ_{y}.wH₂O in which Q represents respectively chlorine, bromine or iodine, (and especially chlorine to form a chlorohydrate) x is variable from 2 to 5 and x+y=6 while wH₂O represents a variable amount of hydration.

Zirconium actives can usually be represented by the empirical general formula: ZrO(OH)_{2n-nz}B_{z}.wH₂0 in which z is a variable in the range of from 0.9 to 2.0 so that the value 2n-nz is zero or positive, n is the valency of B, and B is selected from the group consisting of chlorine (to form a chlorohydrate), other halide, sulphamate, sulphate and mixtures thereof. Possible hydration to a variable extent is represented by wH₂0. Preferably, B represents chlorine and the variable z lies in the range from 1.5 to 1.87. In practice, such zirconium salts are usually not employed by themselves, but as a component of a combined aluminium and zirconium-based antiperspirant.

The above aluminium and zirconium salts may have co-ordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

In some compositions, it is highly desirable to employ complexes of a combination of aluminium chlorohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Giulini, from Summit and from Reheis.

The invention compositions can comprise, if desired, a deodorant active other than an antiperspirant active described hereinbefore. Such an alternative deodorant active can be selected conveniently from any deodorant active known in the cosmetic art such as antimicrobial actives such as polyhexamethylene biguanides, e.g. those available under the trade name Cosmocil™ or chlorinated aromatics, eg triclosan available under the trade name Irgasan™, non-microbiocidal deodorant actives such as triethylcitrate, bactericides and bacteriostatis. Yet other deodorant actives can include bactericidal zinc salts such as zinc ricinoleate. The concentration of such alternative deodorant active is desirably from 0.01 to 5% and in many instances is from 0.1 to 1% by weight of the composition.

In many highly desirable invention compositions, an antiperspirant active is present.

High desirably, the invention emulsions either are at least substantially free from a short chain aliphatic monohydric alcohol, conventionally up to C6, and particularly ethanol. By substantially in this context is meant less than 5% by weight of the composition, preferably less than 3% by weight, particularly less than 1% by weight and more particularly less than 0.5% by weight. Especially preferably, said alcohol and particularly ethanol, is totally absent or at worst less than 0.1% by weight is present.

An essential constituent of compositions of the present invention is a non-ionic emulsifier or mixture of emulsifiers forming an emulsifier system. Such emulsifiers have been found to be compatible with the unusually thin-walled perfume encapsulates also used in the present invention. Such an emulsifier system conveniently has a mean HLB value in the region of from about 5 to about 12 and particularly from 6 to about 10. An especially desired mean HLB value is from 7 m to 9. Such a mean HLB value can be provided by selecting an emulsifier having such an HLB value, or more preferably by employing a combination of at least two emulsifiers, a first (lower) HLB emulsifier having an HLB value in the range of from 2 to 6.5, such as in particular from 4 to 6 and a second (higher) HLB emulsifier having an HLB value in the range of from about 6.5 to 18 and especially from about 12 to about 18. When a combination of emulsifiers is employed, the average HLB value can be obtained by a weight average of the HLB values of the constituent emulsifiers.

An especially desirable range of emulsifiers comprise a hydrophilic moiety provided by a polyalkylene oxide (polyglycol), and a hydrophobic moiety provided by an aliphatic hydrocarbon, preferably containing at least 10 carbons and commonly linear. The hydrophobic and hydrophilic moieties can be linked via an ester or ether linkage, possibly via an intermediate polyol such as glycerol.

Preferably the hydrophobic aliphatic substituent contains at least 12 carbons, and is derivable from lauryl, palmityl, cetyl, stearyl, olearyl and behenyl alcohol, and especially cetyl, stearyl or a mixture of cetyl and stearyl alcohols or from the corresponding carboxylic acids. It is particularly convenient to employ an emulsifier comprising a polyalkylene oxide ether.

The polyalkylene oxide is often selected from polyethylene oxide and polypropylene oxide or a copolymer of ethylene oxide and comprises a polyethylene oxide. The number of alkylene oxide and especially of ethoxylate units within suitable emulsifiers is often selected within the range of from 2 to 100. Emulsifiers with a mean number of ethoxylate units in the region of 2 can provide a lower HLB value of below 6.5 and those having at least 4 such units a higher HLB value of above 6.5 and especially those containing at least 10 ethoxylate units. A preferred combination comprises a mixture of an ethoxylate containing 2 units and one containing from 10 to 40 units. Particularly conveniently, the combination of emulsifiers comprises steareth-2 and a selection from steareth-15 to steareth-30.

It is desirable to employ a mixture of ethoxylated alcohol emulsifiers in a weight ratio of emulsifier having a lower HLB value of <6.5 to emulsifier having a higher HLB value of >8 of from 1.5:1 to 6:1 and particularly from 2:1 to 5:1.

The total proportion of emulsifiers in the composition is usually at least 1.5% and particularly at least 2% by weight. Commonly the emulsifiers are not present at above 6%, often not more than 5% by weight and in many preferred embodiments up to 4% by weight. An especially desirable concentration range for the emulsifiers is from 2.5 to 4% by weight.

Another essential constituent of the present invention compositions is an oil, by which is meant a liquid that is water-immiscible. Such oils are characterised by being liquid at 20° C. (at 1 atmosphere pressure) and are often selected from silicone oils, hydrocarbon oils, ester oils, ether oils and alcohol oils or a mixture of two or more oils selected from such classes of oils. It is highly desirable that the oil has a boiling point of above 100° C. and preferably above 150° C.

The oil is advantageously a plant oil and particularly is a triglyceride oil. Such oils are often obtainable by extraction from the plant's seeds. Suitable plant oils include sunflower seed oil, maize corn oil, evening primrose oil, coriander seed oil, safflower oil, olive oil, rape seed oil, castor oil and borage seed oil. It is particularly desirable to employ an oil which comprises mono or polyunsaturated long chain aliphatic carboxylate substituents, such as notably C18 carboxylates containing 1, 2 or 3 degrees of unsaturation, 2 or more of which may be conjugated. Other suitable oils which come into consideration include jojoba oil.

Alternatively or additionally, the oil can comprise a volatile silicone oil, viz., a liquid polyorgano-siloxane having a measurable vapour pressure at 25° C. of at least 1 Pa, and typically in a range of from 1 or 10 Pa to 2 kPa. Volatile polyorganosiloxanes can be linear or cyclic or mixtures thereof. Preferred cyclic siloxanes, otherwise often referred to as cyclomethicones, include cyclopenta-methicone and hexacyclomethicone, and mixtures thereof.

The ester oil can be aliphatic or aromatic, commonly containing at least one residue containing from 10 to 26 carbon atoms. Examples of suitable aliphatic oils include isopropyl myristate, isopropyl palmitate, myristyl myristate. Preferably the aromatic ester oil is a benzoate ester. Preferred benzoate esters satisfy the formula

Ph—CO—O—R in which R is an aliphatic group containing at least 8 carbons, and particularly from 10 to 20 carbons such as from 12 to 15, including a mixture thereof.

The ether oil preferably comprises a short chain alkyl ether of a polypropylene glygol (PPG), the alkyl group comprising from C2 to C6, and especially C4 and the PPG moiety comprising from 10 to 20 and particularly 14 to 18 propylene glycol units. An especially preferred ether oil bears the INCl name PPG14-butyl ether.

Examples of suitable non-volatile hydrocarbon oils include polyisobutene and hydrogenated polydecene. Examples of suitable non-volatile silicone oils include dimethicones and linear alkylarylsiloxanes. The dimethicones typically have an intermediate chain length, such as from 20 to 100 silicon atoms. The alkylarylsiloxanes are particularly those containing from 2 to 4 silicon atoms and at least one phenyl substituent per silicon atom, or at least one diphenylene group. The aliphatic alcohol desirably is a branched chain monohydric alcohol containing from 12 to 40 carbon atoms, and often from 14 to 30 carbon atoms such as isostearyl alcohol.

Even though, some fragrance oils include an ester group or an ether group, herein the weight of fragrance materials is not included herein in calculating the weight of the oil blend, irrespective of whether the fragrance is encapsulated or “free” (non-encapsulated). The weight of fragrance materials is not included herein in calculating the weight of the oil blend, irrespective of whether the fragrance is encapsulated or “free”.

The proportion of oil in the composition (excluding any contribution from water-insoluble constituents of fragrance oils which may be present) is often at least 1% and commonly a least 1.5% by weight. In many instances the proportion of oil is not more than 10% by weight and notably is not more than 5% by weight.

In many suitable embodiments the total proportion of emulsifier(s) and oils (excluding fragrance oils) is selected in the range of from 4 to 7.5% by weight of the emulsion.

The combination of emulsifiers at suitably chosen concentrations can in many embodiments generate a suitable viscosity to enable the composition to function effectively in a roll-on dispenser. However, if desired, a water-soluble or water-dispersible thickener and/or a particulate insoluble thickener can be employed, to increase the viscosity of the composition, thereby enabling a lower overall concentration of emulsifiers to be employed. Such thickeners include water-soluble or water-dispersible polymers include cellulose derivatives such as starches, carboxymethyl cellulose, ethylcellulose polymers, hydroxyethylcellulose polymers, and cellulose ether polymers, and/or gelatins and/or plant-extract polysaccharides thickeners such as extracts from seaweed. Other effective polymeric thickeners include polyethylene oxide, typically with a molecular weight of at least 100,000, and polyacrylic acid. Particulate thickeners include silicas, optionally surface modified, and clays, such as montmorillonite, bentonite and hectorite. The particulate thickeners are finely divided, commonly having a particle size of below 100 μm. Sufficient of such thickener or thickeners is employed to increase the viscosity of the roll-on composition to the desired value.

A preferred constituent of the composition comprises a particulate silica such as an amorphous silica, eg a fumed silica. It is particularly desirable to employ such a fumed (sometimes called pyrogenic) silica which has been hydrophobically treated. Such materials are commercially available under the name hydrophobic silica. Hydrophobic silicas are obtained by chemically bonding a hydrophobic substituent such as especially a siloxane group onto the surface of the silica, possibly following an intermediate treatment in which the surface of the silica has been rendered hydrophilic. Suitable reactants to generate a hydrophobic substituent include halosilanes and in particular chlorosilanes and methylated silazanes such as hexamethyldisilazane. It is particularly desirable to employ a silica that is capable of thickening an oil such as a plant oil.

Desirably, the silica, such as the fumed silica, and especially the hydrophobic silica has a BET specific surface area of at least 100 m²/g and particularly from 150 to 400 m²/g. The silica comprises very fine particles, fumed silica commonly having a diameter for individual particles of below 40 nm and in many instances at least 99% by weight of below 40 nm. In fumed silica as supplied, some aggregation can occur so that in many embodiments, the supplied silica has an average particle size (diameter) of less than or equal to 1000 nm, preferably less than or equal to 500 nm, i.e. the diameter of the silica particle of average weight. In at least some desirable embodiments, at least 99% by weight of the silica particles, as supplied, are in the range of 10 to 500 nm.

The weight proportion of silica in the formulation is often selected taking into account the desired viscosity of the eventual formulation, together with other attributes such as its effect on the speed of drying of the formulation, its perceived greasiness and/or its perceived stickiness. The weight concentration of silica in the composition is desirably at least 0.2%, often at least 0.3% and in many desirable embodiments is at least 0.5% by weight. Its concentration is commonly not greater than 2%, often not greater than 1.5% and in a number of very desirable formulations is not higher than 1.0%. A preferred weight range of silica concentrations is from 0.6 to 0.8%.

The water content of the composition is commonly selected in the range of from 65 to 93% by weight and often from 70 or 75 to 85% by weight.

The weight ratio of silica to water in the invention emulsions is commonly selected in the range of at least 1:400 up to 1:40, often at least 1:275 and in many instances preferably at least 1:200. It is often convenient to employ a weight ratio of up to 1:75.

In addition to the foregoing essential constituents, it is preferable to include a free fragrance, for example in a proportion of from 0.05 or 0.1 to 4% by weight, and particularly from 0.3 to 2% by weight.

In a number of highly desirable embodiments, the invention compositions comprise, by weight, one or more of:

from 70 to 85% of water;

from 10 to 20% of an antiperspirant active, such as actives described hereinbefore;

from 2.5 to 4.0% of an ethoxylated ether emulsifier or mixture of emulsifiers, preferably having an HLB value of from 7 to 9;

from 1.5 to 4% by weight of a plant oil, such as an unsaturated fatty acid triglyceride;

from 0.5 to 1.0% of a hydrophobic fumed silica

from 0.5 to 2.0% of a coacervated gelatin capsules of fragrance and

from 0.3 to 2% of a free fragrance.

By the selection of the proportions of the above identified constituents within the foregoing disclosed ranges of proportions, it is possible to obtain emulsions having a viscosity which fall within a preferred range of from 1000 to 7000 mPa.s and particularly within 2500 to 5500 mPa.s. Viscosities herein are measured in a Brookfield RVT viscometer equipped with a stirrer TA and Hellipath, rotating at 20 rpm at 25° C. unless otherwise stated. Such emulsions demonstrate a particularly desirable combination of product attributes such as a desirable speed of drying compared with emulsions lacking the particulate silica, superior greasiness and avoidance of excessive stickiness on application and a superior retention of fragrance release by rubbing or impact for long periods on the skin after topical application.

Preferably, the emulsion is made by first preparing separate aqueous and oil mixtures which are brought together before shearing. The aqueous phase commonly contains the antiperspirant active. Where a mixed emulsifier system is employed, it is desirable to incorporate any emulsifier having a low HLB value, particularly of <6.5 into the oil phase and an emulsifier having a high HLB value, particularly of >6.5 into the aqueous phase. The temperature of the respective phases can be raised, where necessary, to accelerate dissolution of the emulsifier, for example to above 50° C.

It is highly desirable to incorporate silica and especially hydrophobic silica, with the aqueous phase.

It is preferable to incorporate any fragrance last of all and shortly before the entire mixture is sheared, especially when either or both phases have been heated so as to accelerate emulsifier dissolution. Though the habits of users vary, commonly a user applies from about 0.2 to 0.4 g of composition to an armpit on each application.

In a further aspect of the present invention, there is provided a method of inhibiting perspiration and/or combating malodour perception comprising applying topically to human skin a composition according to the first aspect of the present invention. Advantageously, the composition is applied to localised areas of the body, such as especially in the underarm, but it also be applied to other occluded body areas, such as at the base of the breasts or on the soles of feet.

The invention composition can also be applied via a wipe or a wrist sweat band. The composition is left in place on the body for an extended period, commonly for a period of up to 24 hours, and in many instances from 5 to 18 hours, as is conventional for antiperspirant compositions and thereafter removed by washing in a conventional manner such as using soap and water or showering using shower gels.

The invention formulations are very suitable for dispensing via a roll-on dispenser, for example any dispenser comprising a bottle having a mouth at one end defining a retaining housing for a rotatable member, commonly a spherical ball or less commonly a cylinder which protrudes above the top wall of the bottle. Examples of suitable dispensers are described in EP1175165 or are invert dispensers such as described in U.S. Pat. No. 6,511,243 or in WO2006/007987 or in WO2006/007991. The bottle mouth is typically covered by a cap, typically having a screw thread that cooperates with a thread on the housing or in an innovative design by a plurality of staggered bayonet/lug combinations. Although in past times the bottle commonly was made from glass with a thermoplastic housing mounted in the mouth of the bottle, most roll-on dispensers are now made entirely from thermoplastic polymers.

Having summarised the invention and described it in more detail, together with preferences, specific embodiments will now be described more fully by way of example only.

### EXAMPLES

The capsules E1 and E2 described herein comprised a shell made from a complex coacervate of gelatin with respectively gum arabic or carboxymethylcellulose, cross-linked with glutaraldehyde. E1 is prepared in accordance with the process of WO2006/056096, but with a higher level of incorporated perfume, and E2 in accordance with the process of U.S. Pat. No. 6,045,835, but again with a higher level of incorporated perfume, and in each instance with conditions controlled to obtain the specific characteristics detailed in Table 1.

TABLE 1

| Characteristic | Capsules E1 | | Capsules E2 | |
|---|---|---|---|---|
| Mean particle size D [4.3] | 48.4 | μm | 50.7 | μm |
| Shell thickness (19 to 38 μm) | 0.3-0.65 | μm | | |
| Shell thickness (25 to 35 μm) | | | 0.25-0.6 | μm |
| Shell thickness calculated at mean | 1.3 | μm | 1.8 | μm |
| particle size | | | | |
| DR (11 to 18 μm) | 40:1-58:1 | | 60:1-100:1 | |
| Hysitron hardness | 4.05 | MPa | 4.88 | MPa |
| Apparent Reduced Elastic Modulus | 24.1 | MPa | 27.5 | MPa |

| | | |
|---|---|---|
| Wt % oils/fragrance in core | 85/40 | 80/80 |

Mean Particle Size: D[4.3] of the capsules after dispersion in volatile silicone (cyclopentadimethicone) was obtained using a Malvern Mastersizer 2000, the following parameters.
RI of Dispersant = 1.397
Dispersion module mixer speed = 2100 rpm.
Result calculation model = General purpose.
Calculation sensitivity = Normal.
Particle shape = Spherical

Shell Thickness: Measured by SEM on encaps with a particle size specified. For non-spherical encaps, the thickness was measured at or close to the minimum encapsulate diameter,

Shell Thickness (Calculated): Calculation assumed that capsules were spherical, with a single core and the shell and core had the same density.

DR is the ratio of av. particle diameter:measured shell thickness.

### Example 1

In this Example, the effectiveness of emulsion antiperspirant compositions containing a floral (Bm) non-encapsulated fragrance and either containing or lacking an encapsulated fragrance product E1 or E2 (containing encapsulated floral-green fragrance) were measured and compared. The compositions are indicated in Table 2.

The effectiveness was determined in the following test in which 24-26 panelists self-applied approximately 0.3 g example stick product to either the left or right armpit and comparison product to the other, with overall left-right randomized balance or an approximately 2 second spray.

After application of the antiperspirant formulations, the users put on their normal clothing and the intensity of the odour was assessed at 2 hourly intervals on a scale of perception increasing from 0 to 10. The scores were averaged and that for the non-encapsulated sample deducted from that for the encapsulated sample. Three scores were measured, namely intensity of the fragrance itself, the intensity detected through the clothing and finally the intensity of any malodour. The results are summarized in Table 3.

TABLE 2

| Ingredient | % by weight | | |
|---|---|---|---|
| Water | Balance | | |
| ACH (50% w/w solution | 30.0 | | |
| Triglyceride oil | 2.0 | | |
| Steareth-2 | 2.3 | | |
| Steareth-20 | 0.9 | | |
| Hydrophobic silica | 0.7 | | |
| Fragrance | E1 | E2 | Bm |
| amount | 1.5 | 0.7 | 1.0 |

The proportion of fragrance employed from E1 and E2 was approximately the same, at about 0.6%.

TABLE 3

| | Difference in Intensity at assessment | | | | | |
|---|---|---|---|---|---|---|
| | Direct | | Through Clothing | | Malodour | |
| Assessment | Bm + E1 v | Bm + E2 v | Bm + E1 v | Bm + E2 v | Bm + E1 | Bm + E2 |
| time Hrs) | Bm | Bm | Bm | Bm | v Bm | v Bm |
| 0 | 0.86 | 0.29 | 0.38 | 0.45 | n/d | n/d |
| 2 | 0.71 | 0.43 | 0.53 | 0.55 | −0.09 | −0.05 |
| 4 | 1.06 | 0.42 | 0.90 | 0.50 | −0.19 | −0.05 |
| 6 | 1.19 | 0.62 | 1.20 | 0.55 | 0.00 | 0.05 |
| 8 | 1.43 | 0.09 | 1.00 | 0.60 | −0.23 | −0.20 |
| 10 | 1.33 | 0.29 | 1.00 | 0.20 | −0.24 | −0.40 |
| 12 | 1.28 | 0.48 | 0.80 | 0.05 | −0.43 | −0.05 |
| 14 | 0.95 | 0 | 0.72 | 0.10 | −0.71 | −0.45 |

The results show that the presence of the encapsulated fragrance tended to show significant improvement in suppressing malodour after the composition had been applied for many hours.

### Example 2

Clinical trials were conducted to demonstrate the difference in malodour suppression between encapsulated (test) and non-encapsulated (control) fragrance applied from a roll-on composition. The formulations employed in Example 2 were the same as those employed in Example 1, except that the non-encapsulated fragrance was a different floral fruity fragrance (Cn).

In this Example, test and control product was applied daily to the underarm of panelists (0.3 g+/−0.03 g) and the panelist carried out normal daily activities until after 5 or 24 hours, the effectiveness of the fragrance was assessed by the trained assessor rubbing the underarm gently with latex-gloved fingers (10 strokes). After 2 minutes, the malodour was assessed, but on a scale of from 0 to 5. This was repeated on 4 days, with the panelist instructed not to wash the underarm or apply any other antiperspirant or deodorant during the trial. The results are indicated in Table 4.

TABLE 4

| Fragrance | | Odour Score | |
|---|---|---|---|
| comparison | After (hr.) | Before shear | After shear |
| Cn + E1 v Cn | 5 | −0.11 | −0.10 |
| | 24 | −0.14 | −0.18 |
| Cn + E2 v Cn | 5 | −0.07 | −0.09 |
| | 24 | −0.12 | −0.22 |

These results confirm that presence of the encapsulated fragrances enabled the composition to control malodour better, even after 24 hours.

### Examples 3 to 8

The compositions in these Examples made by the same general method as for Example 1 are summarised in Table 5 below.

TABLE 5

| | Example no | | | | | |
|---|---|---|---|---|---|---|
| | 3 | 4 | 5 | 6 | 7 | 8 |
| Ingredient | % by weight | | | | | |
| ACH*¹ | 25.0 | 30.0 | 35.0 | 40.0 | 30.0 | 30.0 |
| Steareth-2*² | 2.3 | 2.3 | 2.0 | 2.0 | 2.3 | 2.6 |
| Steareth-20*³ | 0.9 | 0.9 | 0.5 | 0.5 | 0.9 | 0.6 |
| Helianthus Annuus*⁴ | 2.0 | | | | 2.0 | 4.0 |
| Alkyl Benzoate*⁵ | | 2.0 | | | | |
| PPG butyl ether*⁶ | | | 4.0 | | | |
| Cyclomethicone*⁷ | | | | 4.0 | | |
| Hydrophobic Silica*⁸ | 0.7 | 0.3 | 0.7 | | | |
| Fumed Silica*⁹ | | | | | 1.0 | 1.5 |
| E1 | 1.6 | | 1.2 | | 1.5 | |
| E2 | | 1.0 | | 0.8 | | 1.2 |
| Free fragrance | 1.5 | 0.8 | 0.8 | 0.8 | 0.8 | |

| | |
|---|---|
| Water | balance to 100% |

*¹Aluminium Chlorohydrate (50% w/w aqueous solution - Chlorhydrol ™ sol - Reheis
*²Tego Alkanol S2 ™ - Degussa
*³Brij 78 ™ - Uniquema
*⁴high oleic - Henry Lamotte
*⁵Finsolv TN - Finetex
*⁶Fluid AP - Ucon
*⁷Cab-o-sil - Cabot
*⁸HDK H30 ™ - Wacker Chemie

1. An antiperspirant or deodorant composition in the form of an oil-in-water emulsion comprising:
a continuous aqueous phase in which is dissolved or dispersed an antiperspirant or deodorant active;
a dispersed oil phase;
a nonionic emulsifier or mixture of emulsifiers; and
a dispersed particulate shear sensitive encapsulated perfume, wherein the perfume encapsulates have a shell of cross-linked gelatin coacervate having a thickness of from 0.25 to 9 μm and providing from 10 to 40% by weight of the capsules, a volume average particle diameter of from 25 to 70 μm, a ratio of shell thickness to the average particle diameter in the range of from 1:5 to 1:120, and a Hysitron hardness in the range of from 1.5 MPa to 50 MPa.

2. A composition according to claim 1 in which the coacervate is obtained by contacting gelatin with either gum Arabic or a charged carboxymethyl cellulose at a pH of below 5.

3. A composition according to claim 1 in which the coacervate is cross-linked with glutaraldehyde.

4. A composition according to claim 1 in which the capsules have a particle size D[4,3] in the range of from 40 to 60 μm.

5. A composition according to claim 1 in which the capsules have a measured shell thickness in the range of up to 2.5 μm.

6. A composition according to claim 1 in which the capsules have an average measured shell thickness in the range of from 0.3 to 0.8 μm.

7. A composition according to claim 1 in which the capsules have an average particle size:shell thickness ratio in the range of from 40:1 to 80:1.

8. A composition according to claim 1 in which the capsules have an average core volume of from 35 to 55% by volume.

9. A composition according to claim 1 in which the encapsulates have a Hysitron hardness in the range of from 2.5 to 4 MPa.

10. A composition according to claim 1 in which the capsules have an apparent reduced elastic modulus in the range of from 10 to 3 MPa.

11. A composition according to claim 1 which contains from 0.1 to 4% by weight of the capsules.

12. A composition according to claim 1 which additionally contains non-encapsulated fragrance.

13. A composition according to claim 1 in which the emulsifier comprises a mixture of non-ionic emulsifiers, one having an HLB value of from 2 to 6.5 and a second having an HLB value of from 6.5 to 18.

14. A composition according to claim 13 in which the emulsifier comprises a mixture of one emulsifier having an HLB value of <6.5 and the second emulsifier having an HLB value of >8 in a weight ratio of from 2:1 to 5:1.

15. A composition according to claim 1 in which the emulsifier or mixture of emulsifiers is present in an amount together of from 2.5 to 4% by weight of the composition.

16. A composition according to claim 1 in which the oil is present in an amount of at least 1.5% by weight of the composition.

17. A composition according to claim 1 in which the oil is a triglyceride oil.

18. A composition according to claim 1 in which the total proportion of emulsifiers plus oil is in the range of from 4 to 7.5% by weight of the composition.

19. A composition according to claim 1 which contains a fumed silica in an amount of at least 0.5% by weight.

20. A composition according to claim 19 in which the fumed silica is hydrophobic and present in an amount of from 0.5 to 2.0% by weight.

21. A composition according to claim 1 in whch the antipersirant active is an aluminium and/or zirconium chlorohydrate, optionally complexed.

22. A composition according to claim 1 which contains less than 0.1% by weight or no ethanol.

23. A composition according to claim 1 in which the composition contains 70 to 85% by weight of water and 10 to 20% by weight of the antiperspirant active.

24. A composition according to claim 1 in a roll-on dispenser suitable for its application.

25. A process for the manufacture of an antiperspirant or deodorant composition comprising the steps of
separately forming an oil phase containing an emulsifier having an HLB value of <6.5 and an aqueous phase containing an antiperspirant or deodorant active and an emulsifier having an HLB value of >6.5 and optionally containing a thickener for the aqueous phase,
mixing the two phases together and shearing the resultant mixture to form an emulsion,
introducing into the mixture shortly before it is sheared a particulate shear sensitive encapsulated perfume in accordance with claim 1.

26. A method of inhibiting perspiration and/or combating malodour perception comprising applying topically to human skin a composition according to claim 1.
